(19) United States
(12) Patent Application Publication
(10) Pub. No.: US 2010/0063487 A1
(43) Pub. Date: Mar. 11, 2010

(54) LIGHT THERAPY APPARATUS

(76) Inventor: Jason Van Straalen, Mandurah (AU)

Correspondence Address: MERCHANT & GOULD PC, P.O. BOX 2903, MINNEAPOLIS, MN, 55402-0903, US

(21) Appl. No.: 12/088,575

(22) PCT Filed: Sep. 29, 2006
(86) PCT No.: PCT/AU2006/001432

§ 371 Date: Sep. 15, 2008

(30) Foreign Application Priority Data
Sep. 30, 2005 (AU) 2005905403

(51) Int. Cl. A61B 18/18

(52) U.S. Cl. 606/2

(57) ABSTRACT

Apparatus (10) according for treating a patient (12) by applying visible light to the body of the patient for stimulating the skin of the patient and producing a therapeutic effect. The apparatus (10) comprises a body (13) enclosing a treatment station (14) at which the patient (12) can be located in an extended condition. In the extended condition, the patient (12) can be upright or recumbent. In the arrangement where the patient is recumbent, the patient is supported on a bed (15) which is transparent to the light used for treatment purposes. The apparatus (10) is able to dispense light therapy to the entire body in a single treatment session. The apparatus (10) includes a plurality of arrays (16) of light emitting diodes (17) configured to provide a therapeutic treatment to the patient (12). The arrays (16) are each movable along a respective section of the length of the patient to deliver the light therapy such that the arrays in combination deliver light therapy to the body of the patient (12) from head to toe.

### FIELD OF THE INVENTION

The present invention relates to treatment apparatus that provides a therapeutic effect by way of light therapy.

### BACKGROUND ART

A common form of non-surgical face-lift therapy for treating sagging skin and wrinkles on the face operates by stimulating blood supply and thereby increasing muscle tone. This is achieved by subjecting the face to micro-currents for at least one hour. Typically, at least 8 to 10 initial treatments are necessary, followed by monthly maintenance treatments before a desirable effect is present. As a result such non surgical face lift therapy is expensive and time consuming.

Despite it being known that living cells in skin respond to light waves of a particular wavelength, light treatment apparatus are not in wide spread common use. More common forms of treatment include micro-dermabrasion, low level laser therapy and Botox application. These treatment methods suffer from an assortment of problems and deficiencies.

It is against this background, and the problems and deficiencies associated therewith, that the present invention has been developed.

### DISCLOSURE OF THE INVENTION

According to a first aspect of the invention there is provided an apparatus for treating a patient, the apparatus comprising a treatment station at which a patient can be located in an extended condition, at least one array of light emitting elements for stimulating the skin of the patient and producing a therapeutic effect; and means by which the at least one array of light emitting elements are able to apply the therapeutic effect over at least a substantial portion of the length of the patient.

In embodiments, the invention provides a unique method for rapid, efficient and simple treatment of the surface of the skin. The light emitting elements preferably comprise light emitting diodes.

Advantageously, the apparatus treats at least a substantial portion of the entire epidermis of the patient in one session. The apparatus may provide a non-invasive body lift, promoting healthy cells in the skin of the entire body, therefore causing tightness and preferably assisting in the control of the appearance of cellulite in the skin's surface. The light emitting diodes advantageously provide such a overall skin treatment. The whole body is advantageously treated.

In the extended condition, the patient can be in any appropriate position, including upright or recumbent. In the upright position, the patient may be standing and with their arms in any appropriate disposition (such as fully extended upwardly or resting alongside their body). In the recumbent position, the patient may be prostrate, supine or resting on one side, and with their arms in any appropriate disposition (such as resting alongside their body).

Where the patient is in a recumbent position, the patient is preferably elevated.

In one arrangement, there may be light emitting elements disposed on opposed sides of the patient. In such a case there may be two arrays of light emitting elements. Where, for example, the patient is in an elevated condition one array may be disposed above the patient and the other array disposed below the patient.

In another arrangement, the array of light emitting elements may extend substantially around the body of the patient.

Preferably, the patient is supported in the recumbent position on a support surface which is transparent to the light emitted for treatment purposes. The support surface may comprise a clear plastics material such as an acrylic material.

Preferably, the support surface is configured to induce the patient to assume an extended condition in which at least a substantial portion of the entire epidermis of the patient is exposed for treatment. The support surface may, for example, be configured to cause the feet of the patient to tilt downwardly so that the upperside thereof is exposed to one side of the patient's body and the underside is exposed to the other side of the patient's body. In this way, the upperside and underside of the feet are exposed to the light emitted for treatment purposes. The support surface may incorporate foot-wells for this purpose.

The support surface is also preferably contoured to support the patient comfortably.

Preferably, the means for allowing the application of the therapeutic effect comprises means for moving the at least one array of light emitting elements along at least part of the length of the treatment station. Such means may comprise a structure (such as a carriage assembly) upon which the at least one array of light emitting elements is mounted, the structure being operable to move the light emitting diodes between first and second positions.

Means may be provided for sensing the position of the structure relative to the patient and controlling movement thereof accordingly.

Preferred arrangements of the invention are able to rejuvenate the entire surface of the skin of the patient by applying light generated by light emitting elements thereto. The light is preferably applied to the skin in a predetermined manner over a predetermined period of time.

In particular forms of the invention the light emitting diodes are moved over the patient in advantageous patterns of motion. Both linear and circular forms of motion are envisaged.

In particularly preferred embodiments, the body of the apparatus can be opened and closed to provide a chamber into which the person can be received. Preferably the body include a top portion hingedly mounted to a bottom portion.

According to a second aspect of the invention there is provided apparatus for treating a patient, the apparatus comprising a treatment station having a support surface on which a patient can be supported in a recumbent position, means disposed above the treatment station for emitting treatment energy to be directed onto a patient at the treatment station, means disposed below the treatment station for emitting treatment energy to be directed onto the patient at the treatment station, the support surface being transparent to the treatment energy.

According to a third aspect of the invention there is provided apparatus comprising a treatment station having a support surface on which a patient can be supported in a recumbent position, the support surface being configured to induce the patient to assume an extended condition.

According to a fourth aspect of the invention there is provided apparatus comprising a treatment station at which a patient can be located in an extended condition to be treated by application of light thereto, a light source for directing light onto the treatment station, and means for moving the light source along at least part of the length of the treatment station to apply light to at least a portion of the body of the patient located at the treatment station.

According to a fifth aspect of the invention there is provided apparatus comprising a treatment station at which a patient can be located in an extended condition to be treated by application of light thereto, a light source surrounding the treatment station, and means for moving the light source along at least part of the length of the treatment station to apply light to at least a portion of the body of the patient located at the treatment station.

This specification is not to be taken as limited to the above aspects, nor to any preferred feature thereof. Other aspects and preferred features will be apparent from a reading of the specification as a whole.

### BRIEF DESCRIPTION OF THE DRAWINGS

The invention will be better understood by reference to the following description of several specific embodiments thereof as shown in the accompanying drawings in which:

FIG. 1 is a perspective view of an apparatus according to a first embodiment;

FIG. 2 is a schematic sectional side view of the apparatus of FIG. 1 being used to treat a person;

FIG. 3 is a schematic sectional end view of the of FIG. 1 being used to treat the person;

FIG. 4 is a schematic perspective view of one arrangement of cross member carrying an array of light emitting diodes;

FIG. 5 is a schematic perspective view of another arrangement of cross member carrying an array of light emitting diodes;

FIG. 6 is a plan view of the arrangement of light emitting diodes;

FIG. 7 is a perspective view of a control panel incorporating various controls for the environment within the apparatus;

FIG. 8 is a schematic elevational view of apparatus according to a second embodiment, shown on one condition;

FIG. 9 is a view similar to FIG. 8 except that the apparatus is shown in a further condition; and

FIG. 10 is a fragmentary schematic perspective view of an upper ring structure carrying an array of light emitting diodes forming part of the apparatus of the second embodiment.

### BEST MODE(S) FOR CARRYING OUT THE INVENTION

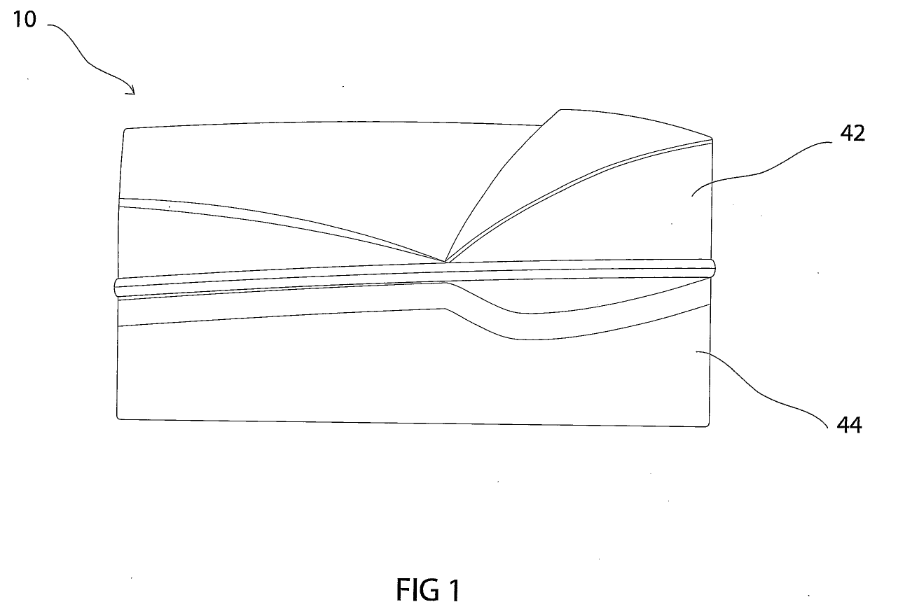

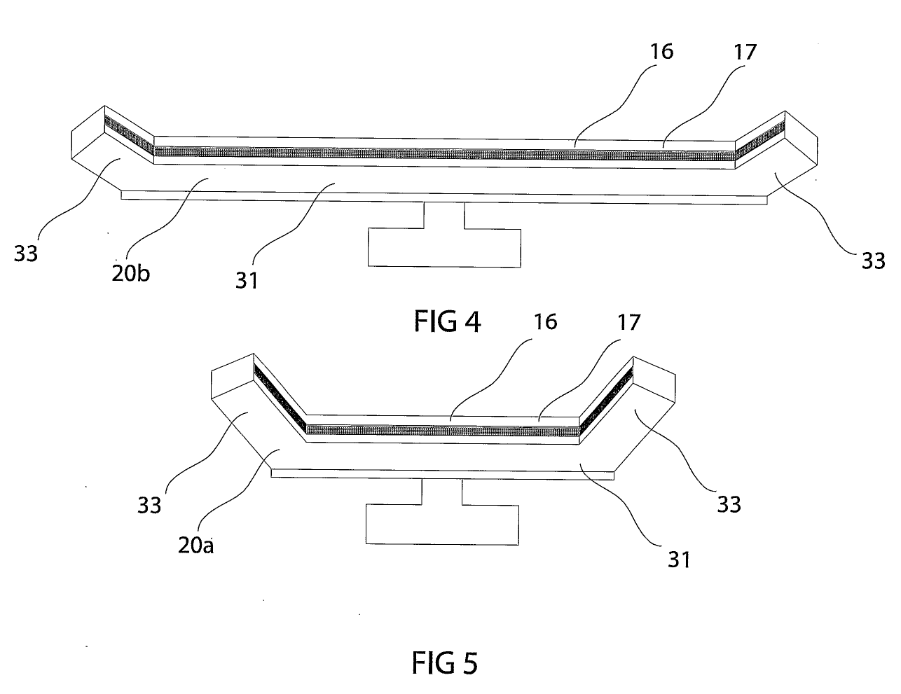

Referring to FIGS. 1 to 7, there is shown an apparatus 10 according to the first embodiment for treating a patient 12 by applying visible light to the body of the patient for stimulating the skin of the patient and producing a therapeutic effect.

The apparatus 10 comprises a body 13 enclosing a treatment station 14 at which there is a support structure configured as a bed 15 adapted to support the patient 12 in recumbent position. The bed 15 is transparent to the light used for treatment purposes and is also elevated.

The apparatus 10 is able to dispense visible light therapy to the entire body of the patient 12 in a single treatment session.

The apparatus 10 includes a plurality of arrays 16 of light emitting diodes 17 configured to provide a therapeutic treatment to the patient 12. Each array typically comprises a multitude of light emitting diodes arranged in a cluster. Typically, the light emitted is visible light.

The arrays 16 include upper arrays disposed above the elevated bed 15 and lower arrays disposed below the elevated bed 15.

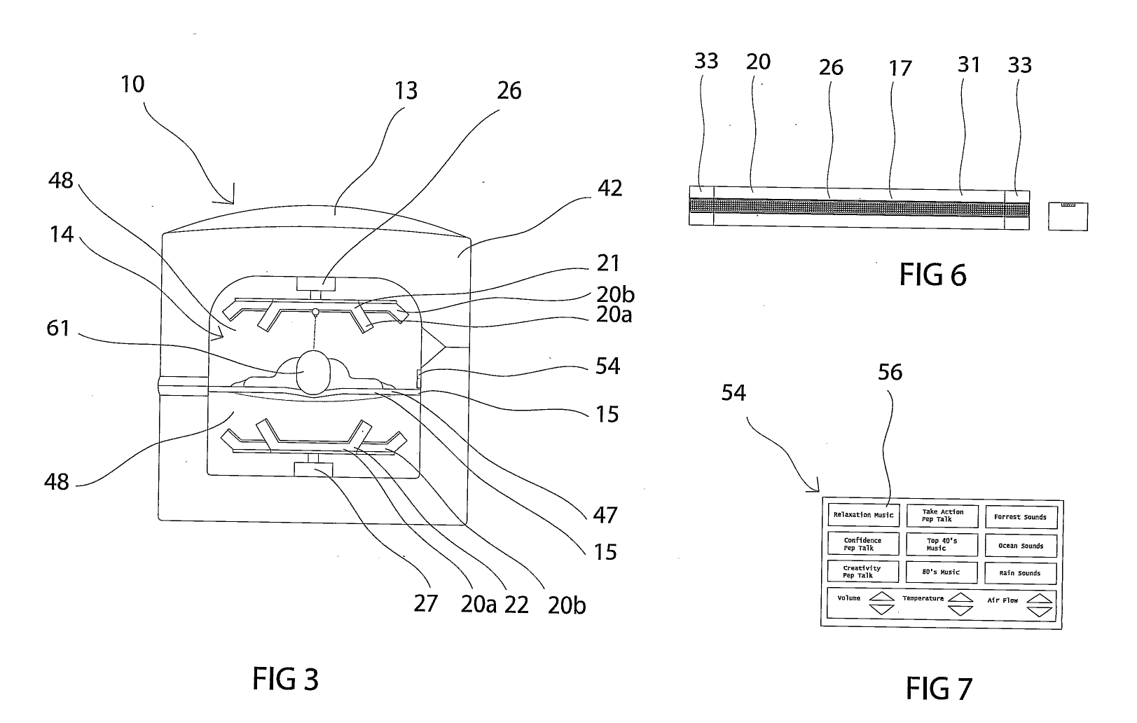

Each array 16 is supported on a cross-member 20 extending laterally with respect to the longitudinal extent of the treatment station 14. In this way, each cross-member 20 extends laterally with respect to the longitudinal extent of patient when in the recumbent position, as best seen in FIG. 3. Accordingly, the cross-members 20 comprise upper cross-members 21 for supporting the upper arrays 16 and lower cross-members 22 for supporting the lower arrays 16. In the arrangement shown, the cross-members 20 are provided in two sizes, one cross-member 20a being smaller and of a size sufficient to span the breadth of the head or lower legs of the patient 12, and the other cross-member 20b being larger and of a size sufficient to span the torso of the patient. With this arrangement, the smaller cross-members 20a are disposed towards the end locations and the larger cross-members 20b are disposed at the intermediate locations.

Each cross-member 20 comprises a central section 31 and two angled end sections 33, the arrangement being such that the cross-member is configured to generally correspond to the cross-sectional profile of the patient, as best seen in FIG. 3.

The apparatus 10 further includes means 23 by which the arrays 16 of light emitting diodes 17 apply the therapeutic effect over the full body length of the patient 12; that is, from head to toe.

In this embodiment, the means 23 comprises an upper frame 24 on which the upper cross-members 21 are mounted in spaced apart relation along the longitudinal extent of the treatment station 14, and a lower frame 25 on which the lower cross-members 22 are also mounted in spaced apart relation along the longitudinal extent of treatment station.

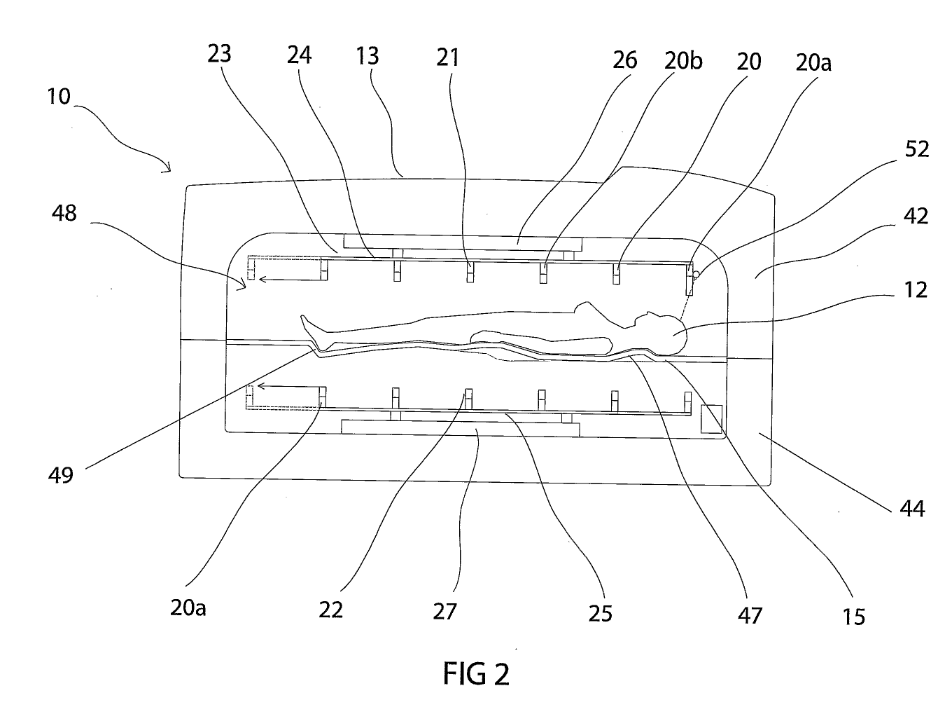

The means 23 further includes an upper carriage mechanism 26 for moving the upper frame 24 in a reciprocatory motion in a direction corresponding to the longitudinal extent of the treatment station 14, and also a lower carriage mechanism 27 for moving the lower frame 25 in a similar way. With this arrangement, the upper and lower frames 24, 25 can be moved back and forth with respect to the patient 12 at the treatment station 14 between two end positions, one of which is shown in solid lines in FIG. 2 and the other of which is shown in phantom lines in that figure. This ensures that the whole length of the body of the patient 12 is exposed to the emitted light during the treatment session.

The body 13 includes a top portion 42 and a bottom portion 44. The top portion 42 and the bottom portion 44 are hingedly mounted together by a hinge (not shown). The bottom portion 44 incorporates the bed 15 which defines a supporting surface 47 at the treatment station 14 for supporting the patient 12 for treatment.

The supporting surface 47 is contoured to support the patient in a comfortable fashion. Further, the supporting surface 47 is configured to induce the patient to assume an extended condition for exposure to the treatment light. Specifically, the supporting surface 47 includes foot-wells 49 configured to cause the feet of the patient to tilt downwardly so that the upperside thereof is exposed to treatment light emitted from above the patient's body and the underside is exposed to treatment light emitted from below the patient's body. In this way, the upperside and underside of the feet are exposed to the light emitted for treatment purposes.

A sensor 52 may be provided for sensing the position of the arrays 16 of light emitting diodes 17 relative to the patient and controlling movement thereof accordingly.

A control panel 54 incorporating various controls 56 is accessible to the patient within the chamber 48 for controlling certain aspects of the environment to which the patient is exposed while within the chamber, including climatic conditions (such as temperature and air flow) and also acoustic conditions (such as the presence of music or other sounds).

In operation, the top portion 42 is lifted upwardly to open the body 13. The patient 12 then climbs into a chamber 48 defined by the body 13 and then lies on the bed 15. Following this the top portion 42 is moved downwardly to close the body 13 and enclose the patient 12 within the chamber 48. This can be performed either by the patient 12, who is now within the chamber 48, or an external operator. The reciprocation of the frames 24, 25 cause the cross-members 20 to reciprocate to move the arrays 16 of light emitting diodes 17 with respect to the patient 12 to provide a therapeutic treatment to the patient, with each array treating a different section of the body of the patient. The treatment may involve a single pass of each array over the respective section of the body of the patient 12 or multiple passes thereover. By controlling movement of the carriage mechanisms 26, 27 and by controlling the light emitting diodes 17, the apparatus 10 advantageously allows for various scanning effects, including circular rotating patterns of therapeutic stimulation as well as linearly varying patterns.

The first embodiment is directed to apparatus 10 for treating a patient resting in a recumbent position, and more specifically in a supine position. The patient can, of course, be supported in any other recumbent position including a prone position and also in a position resting on one side. Other arrangements are also possible, including treatment of a patient in an erect condition such as a standing position.

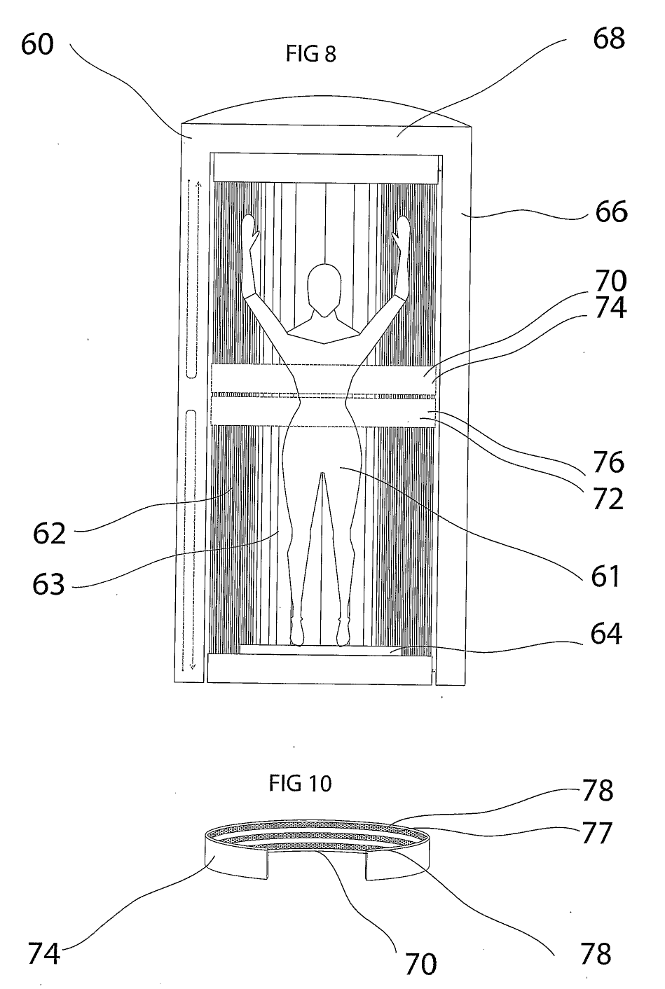

An apparatus 60 according to the second embodiment as shown in FIGS. 8, 9 and 10 is provided for treatment of a patient 61 in a standing position. The apparatus 60 comprises a chamber 62 accommodating a treatment station 63. The chamber 62 has a base 64 on which the patient can stand at the treatment station 63, a side wall 66 and a top 68. A door (not shown) is provided in the side wall 66 for accessing the treatment zone 63.

The apparatus 60 includes an upper array 70 of light emitting diodes and a lower array 72 of light emitting diodes. The upper array 70 is supported on an upper ring structure 74 extending around the treatment zone 63. The lower array 72 is supported on a lower ring structure 76 also extending around the treatment zone 63. Each array 70, 72 comprises light emitting diodes 77 clustered in bands 78, there being three such bands in the arrangement shown.

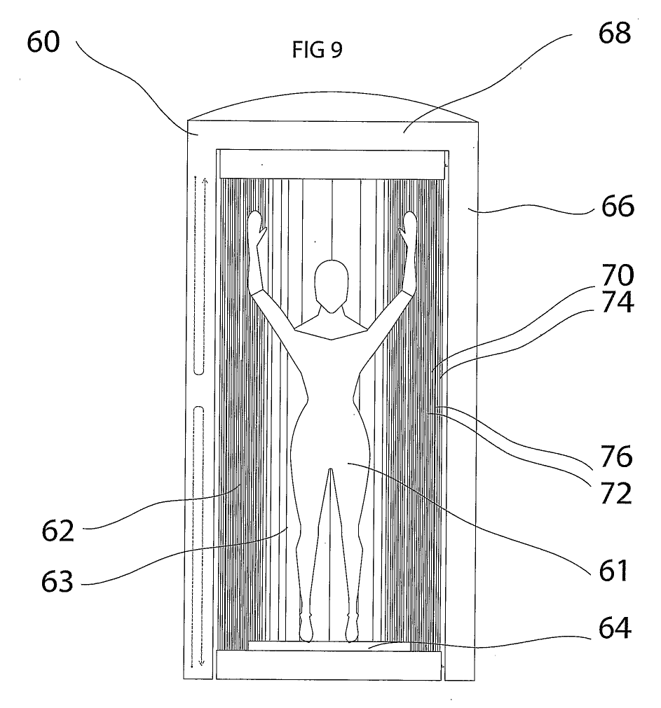

A carrier mechanism (not shown) is provided for moving each ring structure 74, 76 in a reciprocatory fashion upwardly and downwardly so that treatment light is subjected to the entire length of the body of the patient; that is, from head to toe. In this embodiment, the upper ring structure 74 is moveable between a central position adjacent the midriff of the patient (as shown in FIG. 8) to an upper position above the extended arms of the patient (as shown in FIG. 9). Similarly, lower ring structure 76 is moveable between a central position adjacent the midriff of the patient (as shown in FIG. 8) to a lower position below the feet of the patient (as shown in FIG. 9).

The light emitting diodes 77 and carrier mechanism are coupled to a controller (not shown). The controller may be pre-programmed with therapeutic light emitting diodes initiating sequences based on the displacement of the carrier mechanism. The displacement of the carrier mechanism may also be predetermined.

Preferably, the operator programs the controller according to the particular needs of the patient. The treatment may be such that there is a mild body lift promoting healthy cells in the skin of the entire body, therefore causing tightness and even assisting in the control of the appearance of cellulite in the skins surface. This is thought to be particularly advantageous.

It is understood that the low to high range wavelengths of visible light are in the order of 400-750 nm.

In another embodiment, which is not shown, the patient may be supported in a recumbent poison (for example, in an arrangement similar to the first embodiment) and the arrays of light emitting diodes may extend around the patient. The arrays of light emitting diodes may be on ring structures which extend around the treatment station and are moveable along at least a section thereof (for example, in an arrangement similar to that employed in the second embodiment).

It will also be understood that various changes may be made to the form, details, arrangement and proportion of the various parts and steps without departing from the spirit and scope of the invention. Modifications and variations such as would be apparent to the skilled addressee are, at the very least, considered to fall within the scope of the present invention.

Throughout the specification, unless the context requires otherwise, the word “comprise” or variations such as “comprises” or “comprising”, will be understood to imply the inclusion of a stated integer or group of integers but not the exclusion of any other integer or group of integers.

1. Apparatus for treating a patient, the apparatus comprising a treatment station at which a patient can be located in an extended condition, at least one array of light emitting elements for stimulating the skin of the patient and producing a therapeutic effect; and means by which the at least one array of light emitting elements are able to apply the therapeutic effect over at least a substantial portion of the length of the body of the patient.

2. Apparatus according to claim 1 wherein the extended condition of the patient comprises a recumbent position.

3. Apparatus according to claim 2 wherein the station is elevated.

4. Apparatus according to claim 3 wherein there are light emitting elements, disposed on opposed sides of the patient.

5. Apparatus according to claim 4 wherein there are two arrays of light emitting elements, one disposed above the patient and the other disposed below the patient.

6. Apparatus according to claim 1, wherein the array of light emitting elements extends substantially around the body of the patient.

7. Apparatus according to claim 2 wherein the station comprises a support surface transparent to the light emitted for treatment purposes.

8. Apparatus according to claim 7 wherein the support surface is configured to induce the patient to assume an extended condition.

9. Apparatus according to claim 8 wherein the support surface is configured to cause the feet of the patient to tilt downwardly so that the upperside thereof is exposed to one side of the patient's body and the underside is exposed to the other side of the patient's body.

10. Apparatus according to claim 1 wherein the extended condition of the patient comprises a standing position.

11. Apparatus according to claim 10 wherein the array of light emitting elements extends substantially around the body of the patient.

12. Apparatus according to claim 1 wherein the means for allowing the application of the therapeutic effect includes an structure upon which the at least one array of light emitting elements is mounted, the structure being operable to move the light emitting diodes between first and second positions.

13. Apparatus according to claim 12 wherein the structure includes a carriage assembly.

14. Apparatus according to claim 12 further comprising means for sensing the position of the structure relative to the patient and controlling movement thereof accordingly.

15. Apparatus for treating a patient, the apparatus comprising a treatment station having a support surface on which a patient can be supported in a recumbent position, means disposed above the treatment station for emitting treatment energy to be directed onto a patient at the treatment station, means disposed below the treatment station for emitting treatment energy to be directed onto the patient at the treatment station, the support surface being transparent to the treatment energy.

16. Apparatus according to claim 15 wherein the treatment energy comprises light.

17. Apparatus according to claim 16 wherein the light is generated by light emitting elements.

18. Apparatus according to claim 15, wherein the support surface is configured to induce the patient to assume an extended condition.

19. Apparatus according to claim 18 wherein the support surface is configured to cause the feet of the patient to tilt downwardly so that the upperside thereof is exposed to one side of the patient's body and the underside is exposed to the other side of the patient's body.

20. Apparatus for treating a patient, the apparatus comprising a treatment station having a support surface on which a patient can be supported in a recumbent position, the support surface being configured to induce the patient to assume an extended condition.

21. Apparatus according to claim 20 wherein the support surface is configured to cause the feet of the patient to tilt downwardly so that the upperside thereof is exposed to one side of the patient's body and the underside is exposed to the other side of the patient's body.

22. Apparatus comprising a treatment station at which a patient can be located in an extended condition to be treated by application of light thereto, a light source for directing light onto the treatment station, and means for moving the light source along at least part of the length of the treatment station to apply light to at least a portion of the body of the patient located at the treatment station.

23. Apparatus comprising a treatment station at which a patient can be located in an extended condition to be treated by application of light thereto, a light source surrounding the treatment station, and means for moving the light source along at least part of the length of the treatment station to apply light to at least a portion of the body of the patient located at the treatment station.

24. Apparatus according to claim 22 wherein the light source comprises at least one array of light emitting elements.

25. Apparatus according to claim 24 wherein the at least one array of light emitting elements is mounted on a ring structure extending around the treatment station and moveable along at least part of the length thereof.

26. Apparatus according to claim 22 wherein there are at least two light sources each adapted for movement along a respective part of the treatment station.

27. (canceled)
